(19) United States
(12) Patent Application Publication
(10) Pub. No.: US 2004/0146563 A1
(43) Pub. Date: Jul. 29, 2004

(54) Granulated product and process for producing tablets

(76) Inventors: Nobuaki Hirai, Tokyo (JP); Kazuyuki Ishikawa, Tokyo (JP)

Correspondence Address: Peter L Berger, Levisohn Lerner Berger & Langsam, 19th Floor, 805 Third Avenue, New York, NY, 10022, US

(21) Appl. No.: 10/476,275

(22) PCT Filed: Apr. 24, 2002
(86) PCT No.: PCT/JP02/04063

(30) Foreign Application Priority Data
Apr. 27, 2001 (JP) 2001-132476

(51) Int. Cl. A61K 9/14; A61K 35/78

(52) U.S. Cl. 424/489; 424/725

(57) ABSTRACT

Special calcium silicate and crosslinked polyvinylpyrrolidone are fed into a vessel and then a definite amount of water is added thereto. Next, the contents are agitated with an agitator attached to the vessel so that water is uniformly dispersed in the mixture of the special calcium silicate and crosslinked polyvinylpyrrolidone and is retained therein. Then the mixture is granulated while displacing the water in the mixture to a Chinese orthodox medicine extract, a crude drug extract, or an extract derived from a natural substance. Thus, a granulated product having a high content of the Chinese-orthodox medicine extract, the crude drug extract, or the extract derived from a natural substance can be obtained, which makes it possible to miniatuarize or reduce number or amount of doses of tablets each containing the Chinese orthodox medicine extract, the crude drug extract, or the extract derived from a natural substance.

### TECHNICAL FIELD

The present invention relates to a production method for a granulated product containing a Chinese orthodox medicine extract, a crude drug extract, or an extract derived from a natural substance and to a production method for a tablet containing a Chinese orthodox medicine extract, a crude drug extract, or an extract derived from a natural substance, the tablet being produced from the granulated product.

### BACKGROUND ART

As granulation methods for medicine, there are known a dry process pulverization granulation method, a wet process extrusion granulation method, a fluidized bed granulation method, and a high-speed agitation granulation method. Of those, the fluidized bed granulation method and the high-speed agitation granulation method have been widely adopted in pharmaceutical industry since the methods are simpler in process and can produce granulated products more efficiently than other methods.

The fluidized bed granulation method is adopted when granulated products are produced, which contain a Chinese orthodox medicine extract, a crude drug extract, or an extract derived from a natural substance. However, if the products having high contents of such extracts are produced, a decreased fluidity due to high water absorbability of the extracts makes it difficult to perform efficient granulation.

JP 05-95988 A describes a method of improving the fluidized bed granulation method. However, the method uses a special machine (a fluidized bed granulating machine of the floating vortical flow type), and hence it is hard to say that the method has a high general versatility.

On the other hand, among the agitation granulation methods, use of a usual water charging method makes granulation difficult since the extracts rapidly absorb water to become massive or muddy.

There is known a method in which a granulated product containing a Chinese orthodox medicine extract, a crude drug extract, or an extract derived from a natural substance is produced by high speed agitation granulation using calcium silicate. In the method, those extracts and calcium silicate are mixed using a high-speed agitation granulating machine (for example, trade name: Vertical Granulator), and water is sprayed onto the resultant mixture (JP 2000-119190 A).

However, in order to prevent rapid absorption of water by the extracts, it is necessary to spray water at an extremely low speed, causing a problem in that granulation is time-consuming. Further, when a different kind of extract is used, it is necessary to change the speed of spraying and granulation time, with the result that it is difficult to find out optimal conditions.

Further, there is a method in which after special calcium silicate is allowed to retain water, granulation is performed using a Chinese orthodox medicine extract or a crude drug extract.

However, even by this method, it is necessary to change the amount of water to be retained by the special calcium silicate and granulation time when the kind of the Chinese orthodox medicine extract or of the crude drug extract is changed.

There has been demanded a granulation method which enables efficient granulation of various extracts not so largely dependent on the scale of production.

Further, the production of a granulated product having a high content of a Chinese orthodox medicine extract, a crude drug extract, or an extract derived from a natural substance is extremely effective in order to make it possible to miniaturize or reduce number or amount of doses of solid tablets each containing a Chinese orthodox medicine extract, a crude drug extract, or an extract derived from a natural substance. Therefore, the production of such a granulated product is an important problem for making dosing easy.

Accordingly, an object of the present invention is to provide a more practical granulation method of producing a granulated product having a high content of a Chinese orthodox medicine extract, a crude drug extract, or an extract derived from a natural substance using a high speed agitation granulating machine and to enable production of tablets compounded each having a high disintegration capability with a high content of a Chinese orthodox medicine extract, a crude drug extract, or an extract derived from a natural substance from the granulated product.

### DISCLOSURE OF THE INVENTION

In the present invention, it has been found that a mixture of a kind of calcium silicate, i.e., a special calcium silicate having a petaloid crystal structure, and a disintegrant can retain more water than the special calcium silicate alone does and undergo milder migration of moisture into the extract upon granulation than the special calcium silicate alone does. Then, a production method for a granulated product containing a Chinese orthodox medicine extract, a crude drug extract, or an extract derived from a natural substance by a novel agitation granulation method that enables production in amounts of from several hundreds grams to several hundreds kilograms has been accomplished.

Hereinafter, description will be made of the agitation granulation method for a Chinese orthodox medicine extract, a crude drug extract, or an extract derived from a natural substance according to the present invention.

First, in the method, the special calcium silicate and a portion or whole of a disintegrant to be used are charged into a vessel. After uniform agitation, a fixed amount of water is charged into the vessel.

Then, the method includes: a humidifying step for agitating the mixture of the special calcium silicate and the disintegrant and water so that water can be uniformly dispersed in the mixture; and a granulation step for charging a Chinese orthodox medicine extract, a crude drug extract, or an extract derived from a natural substance into the mixture of the special calcium silicate and the disintegrant, the mixture retaining water, and granulating the resultant while transferring water retained in the mixture of the calcium silicate and the disintegrant to the Chinese orthodox medicine extract, the crude drug extract, or the extract derived from a natural substance.

The time required for the above-mentioned steps is from 3 to 20 minutes. In the humidifying step, more water can be used according to the present invention than the conventional method in which water is dispersed and retained in the special calcium silicate alone, with the result that stable granulation that does not depend on the production scale is possible.

The agitation granulation in the present invention corresponds to a rolling granulation method in which granulation is performed by charging water or a solution containing a binder and a wet process pulverization granulation method out of the granulation methods described in the item of powder and tablet in general provisions on pharmaceuticals in Commentary on Thirteenth Amendment Japan Pharmacopoeia (an extrusion granulation method, a rolling granulation method, a fluidized bed granulation method, wet process and dry process pulverization granulation methods).

Specifically, this is a wet process granulation method based on a principle on which a powder to be granulated is charged into a vessel, water or a solution containing a binder is charged, and then, the resultant is agitated by a device provided to the vessel.

The agitation method includes those of various modes, including one in which agitation is performed by a rotary arm from above the vessel, one in which agitation is performed by a rotary vane on the bottom of the vessel, and further one in which agitation in different direction is applied as well.

The device used that is commercially available at present includes the following commercial products. That is, Vertical Granulator, High Shear Mixer, High Speed Mixer, Planetary Mixer, Chopper Type Planetary Mixer and so forth.

It has now been found that special calcium silicate having a petaloid crystal structure with a large pore diameter and pore volume is desirably used as the calcium silicate in the present invention. Therefore, the calcium silicate used in the present invention is a special calcium silicate that is distributed under a trade name of “FLORITE-RE” manufactured by Eizai Co., Ltd.

Note that the above-mentioned special calcium silicate meets the standard of chemical name “calcium silicate” described in Japanese Pharmaceutical Excipients (Yakutenki) and Japanese Cosmetic Ingredients Codex (Shougaiki).

The Chinese orthodox medicine extract used in the present invention includes, for example, a Chinese orthodox medicine extract powder obtained by infusing usual Chinese orthodox medicine formulation described in “Guideline for General Chinese Orthodox Medicine Formulation (edited by Pharmaceutical Affairs Bureau—Ministry of Health and Welfare, published by Yakujijiho Co., Ltd. (1975)) with water or an aqueous solution containing 30 wt % or less of ethanol, condensing and drying. Specifically, mention may be made of extract powders of Sho-saiko-to, Sairei-to, Hochu-ekki-to, Saiboku-to, Gosha-jinki-gan, Kami-shoyo-san, Bakumondo-to, Hachimi-jio-gan, Dai-kenchu-to, Sho-seiryu-to, Rikkunshi-to, Toki-shakuyaku-san, Juzen-taiho-to, Kakkon-to, Saiko-keishi-to, Keishi-bukuryo-gan, Choto-san, Dai-saiko-to, Saiko-ka-ryukotsu-borei-to, Chorei-to, Unkei-to, Oren-gedoku-to, Boi-ogi-to, Gorei-san, Byakko-ka-ninjin-to, Shakuyaku-kanzo-to, Hange-byakujutsu-temma-to, Ninjin-yoei-to, Bofu-tsusho-san, Hange-shashin-to, Sho-saiko-to-ka-kikyo-sekko, Keishi-ka-jutsu-bu-to, Keigai-rengyo-to, Hange-koboku-to, Kami-kihi-to, Unsei-in, Seihai-to, Daio-kanzo-to, Jumi-haidoku-to, Toki-inshi, Shin'i-seihai-to, Toki-shigyaku-ka-goshuyu-shokyo-to, Mao-bushi-saishin-to, Otsuji-to, Kakkon-to-ka-senkyu-shin'i, Anchu-san-ryo, Shofu-san, Keishi-ka-ryukotsu-borei-to, Mao-to, Ninjin-to, Ryo-kei-jutsu-kan-to, Keishi-to, Ma-kyo-kan-seki-to, Seijo-bofu-to, Tokaku-joki-to, Sho-kenchu-to, Keishi-ka-shakuyaku-to, Kikyo-to, Shigyaku-san, Sansonin-to, Keishi-bukuryo-gan-ryo-ka-yokuinin, Ji-zuso-ippo, Shichimotsu-koka-to, Chikujo-untan-to, Shimpi-to, Goko-to, and the like, which are Chinese orthodox medicines.

The crude drug extract powders include those obtained by infusing one or more crude drugs with water or an aqueous solution containing 30 wt % or less of ethanol, condensing and drying. Specifically, mention may be made of Paeoniae Radix, Asparagi Radix, Cinnamomi Cortex, Cnidii Rhizoma, Atractylodis Lanceae Rhizoma, Poria, Moutan Cortex, Spruce, Cyperi Rhizoma, Rehmanniae Radix, Glycyrrhizae Radix, Persicae Semen, Coptidis Rhizoma, Zingiberis Rhizoma, Caryophylli Flos, Ginseng Radix, Aurantii Nobilis Pericarpium, Corydalis Tuber, Valerian, Aurantii Fructus Immaturus, and Arctii Fructus.

The extract powders derived from natural substances include those obtained by infusing natural substances with water or an aqueous solution containing 30 wt % or less of ethanol, condensing and drying. Specifically, mention may be made of Saw palmetto, Silybum marianum, Turmeric, Garlic, Aloe, St John's Wort, Ginkgo, and Echinacea.

Potential disintegrants include potato starch, crystalline cellulose, carboxymethyl cellulose, carboxymethyl cellulose calcium, low-substituted hydroxypropyl cellulose, crosscalmelose sodium, carboxymethyl starch sodium, and crosslinked polyvinylpyrrolidone. Preferably, crosslinked polyvinylpyrrolidone is used.

Hereinafter, description will be made of charging amounts of water, Chinese orthodox medicine extract powder, crude drug extract powder, extract powder derived from a natural substance (hereinafter referred to as extract powders), special calcium silicate, and disintegrant upon producing granulated products each containing a Chinese orthodox medicine extract, a crude drug extract, or an extract derived from a natural substance in the present invention.

The compounding amount of the disintegrant is determined based on the amounts of the Chinese orthodox medicine extract powder, the crude drug extract powder, or the extract powder derived from a natural substance, and the special calcium silicate.

Usually, the compounding amount is 1 to 50 parts by weight, preferably 7 to 30 parts by weight, based on 10 parts by weight of the special calcium silicate.

The amount of water is preferably 2 to 50 parts by weight, more preferably 5 to 25 parts by weight, based on 10 parts by weight of the special calcium silicate. If the amount of water to be charged is little, no grain is formed in the step of charging extract powder, which is a downstream operation. On the other hand, if the amount of water is too much, granulation proceeds too much, so that the machine cannot be operated.

Instead of water, an aqueous solution containing 30 wt % or less of ethanol may be used. If the amount of the aqueous ethanol solution to be used is little, no grain is formed in the step of charging the extract powder, which is a downstream operation. On the other hand, if the amount of the aqueous ethanol solution is too much, granulation proceeds too much, so that the machine cannot be operated.

The compounding amount of the extract powder is usually 1 to 100 parts by weight, preferably 40 to 80 parts by weight, based on 10 parts by weight of the special calcium silicate. That is, the extract-compounding amount in the finished granulated product is usually 30 to 90 wt %, preferably 60 to 75 wt %. If a ratio of the Chinese orthodox medicine extract, the crude drug extract, or the extract derived from a natural substance is high, granulation becomes impossible.

In the case where tablets are produced from the granulated product containing the Chinese orthodox medicine extract, the crude drug extract, or the extract derived from a natural substance, the charging amount of the granulated product is determined based on the amount of the extract daily dosed and on the number of tablets daily dosed necessary for exhibiting therapeutic efficacy or effect.

If the ratio of the extract is little, the number of tablets manufactured becomes larger or the extract sufficient for exhibiting efficacy cannot be formulated when tablets are manufactured.

The above-mentioned granulated product may be compounded with an excipient having the following components as an additive.

The excipient includes: lactose, white sugar, glucose, mannitol, sorbitol and so forth among those classified into sugars and sugar alcohols; corn starch, potato starch, α-converted starch, dextrin, carboxymethyl starch and so forth among those classified into starch and starch derivatives; crystalline cellulose, hydroxypropyl cellulose, carboxymethyl cellulose and so forth among those classified into cellulose and cellulose derivatives; and as others gum Arabic, dextran, pullulan, light silicic anhydride, synthetic aluminum silicate, magnesium metasilicate aluminate, calcium phosphate, calcium carbonate, calcium sulfate and so forth.

In the case where tablets are produced from the above-mentioned granulated product, the granulated product is subjected to regularization and then a lubricant is added as an additive thereto and the whole is mixed to produce granules for tableting. In this case, a disintegrant may be added as necessary.

Potential disintegrants include potato starch, crystalline cellulose, carboxymethyl cellulose, carboxymethyl cellulose calcium, low-substituted hydroxypropyl cellulose, crosscalmelose sodium, carboxymethyl starch sodium, and crosslinked polyvinylpyrrolidone. Preferably, crosslinked polyvinylpyrrolidone is used.

The compounding amount of the disintegrant or lubricant in one tablet is determined based on the amounts of the extract powder, calcium silicate, and the disintegrant in the granulated product.

Potential lubricants include metal stearate (magnesium, calcium), talc, hardened castor oil, and sodium stearyl fumarate. The compounding amount of the lubricant is 0.1 to 5 wt %, desirably 0.3 to 2 wt % of the weight of one tablet.

Tableting granules for production are converted into tablets using a tableting machine. Note that the method of converting granules for tableting into tablets is similar to conventional one and explanation of the method is omitted.

The method of the present invention may include a coating step for applying a film coating or sugar coating to the thus produced tablets. Note that by application of the film coating or sugar coating, masking of the taste of the tablet becomes possible, so that the tablets can be made easy to take. Further, the storage stability of the tablets is increased. Note that in the case of the tablet of the present invention, the step and raw material used are not limited except that an enteric coating and delayed release coating are not adopted.

In the production method according to the present invention, six each of tablets containing Chinese orthodox medicine extracts shown below are formed and their disintegration times were measured according to the disintegration test method described in Japan Pharmacopoeia.

The amount (wt %) of extract in the tested tablet and the obtained disintegration time (minute) are shown. The disintegration time is an average of six tablets tested. The tested tablets include Sho-saiko-to (65 wt %, 7 minutes), Sairei-to (70 wt %, 9 minutes), Hochu-ekki-to (70 wt %, 8 minutes), Saiboku-to (70 wt %, 10 minutes), Gosha-jinki-gan (65 wt %, 9 minutes), Kami-shoyo-san (70 wt %, 9 minutes), Bakumondo-to (75 wt %, 10 minutes), Hachimi-jio-gan (70 wt %, 5 minutes), Dai-kenchu-to (70 wt %, 6 minutes), Sho-seiryu-to (73 wt %, 7 minutes), Rikkunshi-to (70 wt %, 10 minutes), Toki-shakuyaku-san (66 wt %, 8 minutes), Juzen-taiho-to (75 wt %, 9 minutes), Kakkon-to (70 wt %, 10 minutes), Saiko-keishi-to (70 wt %, 9 minutes), Keishi-bukuryo-gan (70 wt %, 10 minutes), Choto-san (70 wt %, 6 minutes), Dai-saiko-to (70 wt %, 8 minutes), Saiko-ka-ryukotsu-borei-to (75 wt %, 12 minutes), Chorei-to (65 wt %, 6 minutes), Unkei-to (70 wt %, 9 minutes), Oren-gedoku-to (65 wt %, 7 minutes), Boi-ogi-to (70 wt %, 8 minutes), Gorei-san (65 wt %, 7 minutes), Byakko-ka-ninjin-to (75 wt %, 12 minutes), Shakuyaku-kanzo-to (60 wt %, 8 minutes), Hange-byakujutsu-temma-to (75 wt %, 8 minutes), Ninjin-yoei-to (70 wt %, 10 minutes), Bofu-tsusho-san (70 wt %, 7 minutes), Hange-shashin-to (72 wt %, 9 minutes), Sho-saiko-to-ka-kikyo-sekko (65 wt %, 8 minutes), Keishi-ka-jutsu-bu-to (70 wt %, 7-minutes), Keigai-rengyo-to (70 wt %, 10 minutes), Hange-koboku-to (70 wt %, 10 minutes), Kami-kihi-to (75 wt %, 10 minutes), Unsei-in (70 wt %, 8 minutes), Seihai-to (70 wt %, 12 minutes), Daio-kanzo-to (70 wt %, 8 minutes), Jumi-haidoku-to (70 wt %, 8 minutes), Toki-inshi (65 wt %, 9.5 minutes), Shin'i-seihai-to (75 wt %, 12 minutes), Toki-shigyaku-ka-goshuyu-shokyo-to (75 wt %, 12 minutes), Mao-bushi-saishin-to (65 wt %, 6 minutes), and Otsuji-to (65 wt %, 9 minutes).

As a result, it has been revealed that although disintegration time may differ depending on the Chinese orthodox medicine extract used, the tablets are disintegrated at most within 15 minutes.

### BEST MODES FOR CARRYING OUT THE INVENTION

Hereinafter, best modes for carrying out the present invention will be described.

In the best modes for carrying out the present invention, the agitation rotation speeds upon granulation were about 200 rpm for a bottom agitation device (blade) and about 3,000 rpm for an agitation device in a direction different to the direction of the bottom agitation device (cross-screw).

Embodiment 1

A granulated product containing Hachimi-jio-gan extract powders serving as the Chinese orthodox medicine extract was produced as follows.

10 parts by weight of calcium silicate (trade name: FLORITE-RE) and 14.6 parts by weight of crosslinked polyvinylpyrrolidone (trade name: Kollidon CL) were sifted and charged into Vertical Granulator (granulating machine). After that, 17.5 parts by weight of water was added thereto and mixed.

Then, 60 parts by weight of Hachimi-jio-gan extract powder was added and the resultant was subjected to agitation granulation for 3 minutes. After the granulation, the resultant was subjected to regularization and dried.

Further, 0.4 part by weight of magnesium stearate was added to form granules for tableting, and circular tablets were produced from the granules using a tableting machine.

The tablets were subjected to the disintegration test described in Japan Pharmacopoeia. As a result, disintegration times for the six tablets were 3.7 minutes, 4.0 minutes, 4.7 minutes, 4.7 minutes, 5.1 minutes, and 5.2 minutes, respectively, with the average of the six tablets being 4.6 minutes.

### COMPARATIVE EXAMPLE 1

In Comparative Example 1 for Embodiment 1, commercially available Hachimi-jio-gan extract tablets were subjected to the disintegration test described in Japan Pharmacopoeia. As a result, average disintegration times for the six tablets were as follows.

Product A: 32 minutes

Product B: 19 minutes.

Embodiment 2

A tablet containing extract of Hachimi-jio-gan serving as the Chinese orthodox medicine was produced as follows.

10 parts by weight of calcium silicate (trade name: FLORITE-RE) and 2.2 parts by weight of crosslinked polyvinylpyrrolidone (trade name: Kollidon CL) were sifted and charged into Vertical Granulator (granulating machine). After that, 17.5 parts by weight of water was added thereto and mixed.

Then, 60 parts by weight of Hachimi-jio-gan extract powder was added and the resultant was subjected to agitation granulation for 3 minutes. After the granulation, the resultant was dried and subjected to regularization.

Further, 12.4 parts by weight of crosslinked polyvinylpyrrolidone (trade name: Kollidon CL) and0.4part by weight of magnesium stearate were added to form granules for tableting, and circular tablets were produced from the granules using a tableting machine.

The tablets were subjected to the disintegration test described in Japan Pharmacopoeia. As a result, disintegration times for the six tablets were 6.6 minutes, 10.7 minutes, 12.1 minutes, 12.7 minutes, 13.1 minutes, and 13.9 minutes, respectively, with the average of the six tablets being 11.6 minutes.

Embodiment 3

A tablet containing extract of Hachimi-jio-gan serving as the Chinese orthodox medicine was produced as follows.

10 parts by weight of calcium silicate (trade name: FLORITE-RE) and 4.4 parts by weight of crosslinked polyvinylpyrrolidone (trade name: Kollidon CL) were sifted and charged into Vertical Granulator (granulating machine). After that, 17.5 parts by weight of water was added thereto and mixed.

Then, 60 parts by weight of Hachimi-jio-gan extract powder was added and the resultant was subjected to agitation granulation for 3 minutes. After the granulation, the resultant was dried and subjected to regularization.

Further, 10.2 parts by weight of crosslinked polyvinylpyrrolidone (trade name: Kollidon CL) and 0.4 part by weight of magnesium stearate were added to form granules for tableting, and circular tablets were produced from the granules using a tableting machine.

The tablets were subjected to the disintegration test described in Japan Pharmacopoeia. As a result, disintegration times for the six tablets were 6.6 minutes, 7.6 minutes, 8.6 minutes, 8.6 minutes, 9.1 minutes, and 10.7 minutes, respectively, with the average of the six tablets being 8.5 minutes.

Embodiment 4

A tablet containing extract of Hachimi-jio-gan serving as the Chinese orthodox medicine was produced as follows.

10 parts by weight of calcium silicate (trade name: FLORITE-RE) and 6.6 parts by weight of crosslinked polyvinylpyrrolidone (trade name: Kollidon CL) were sifted and charged into Vertical Granulator (granulating machine). After that, 17.5 parts by weight of water was added thereto and mixed.

Then, 60 parts by weight of Hachimi-jio-gan extract powder was added and the resultant was subjected to agitation granulation for 3 minutes. After the granulation, the resultant was dried and subjected to regularization.

Further, 8.0 parts by weight of crosslinked polyvinylpyrrolidone (trade name: Kollidon CL) and 0.4 part by weight of magnesium stearate were added to form granules for tableting, and circular tablets were produced from the granules using a tableting machine.

The tablets were subjected to the disintegration test described in Japan Pharmacopoeia. As a result, disintegration times for the six tablets were 5.9 minutes, 6.2 minutes, 6.6 minutes, 7.2 minutes, 7.6 minutes, and 8.1 minutes, respectively, with the average of the six tablets being 6.9 minutes.

Embodiment 5

A tablet containing extract of Hachimi-jio-gan serving as the Chinese orthodox medicine was produced as follows.

10 parts by weight of calcium silicate (trade name: FLORITE-RE) and 10.8 parts by weight of crosslinked polyvinylpyrrolidone (trade name: Kollidon CL) were sifted and charged into Vertical Granulator (granulating machine). After that, 22.5 parts by weight of water was added thereto and mixed.

Then, 60 parts by weight of Hachimi-jio-gan extract powder was added and the resultant was subjected to agitation granulation for 18 minutes. After the granulation, the resultant was dried and subjected to regularization.

Further, 0.16 part by weight of magnesium stearate was added to form granules for tableting, and circular tablets were produced from the granules using a tableting machine.

The tablets were subjected to the disintegration test described in Japan Pharmacopoeia. As a result, disintegration times for the six tablets were 4.9 minutes, 5.8 minutes, 6.4 minutes, 9.3 minutes, 9.9 minutes, and 10.6 minutes, respectively, with the average of the six tablets being 7.8 minutes.

Embodiment 6

A tablet containing extract of Hachimi-jio-gan serving as the Chinese orthodox medicine was produced as follows.

10 parts by weight of calcium silicate (trade name: FLORITE-RE) and 10.8 parts by weight of carboxymethyl starch sodium (trade name: Explotab) were sifted and charged into Vertical Granulator (granulating machine). After that, 22.5 parts by weight of water was added thereto and mixed.

Then, 60 parts by weight of Hachimi-jio-gan extract powder was added and the resultant was subjected to agitation granulation for 9 minutes. After the granulation, the resultant was dried and subjected to regularization.

Further, 0.16 part by weight of magnesium stearate was added to form granules for tableting, and circular tablets were produced from the granules using a tableting machine.

The tablets were subjected to the disintegration test described in Japan Pharmacopoeia. As a result, disintegration times for the six tablets were 22.8 minutes, 24.5 minutes, 24.9 minutes, 25.2 minutes, 26.1 minutes, and 26.2 minutes, respectively, with the average of the six tablets being 24.5 minutes.

Embodiment 7

A tablet containing extract of Hachimi-jio-gan serving as the Chinese orthodox medicine extract was produced as follows.

10 parts by weight of calcium silicate (trade name: FLORITE-RE) and 10.8 parts by weight of α-converted starch (trade name: Starch 1500G) were sifted and charged into Vertical Granulator (granulating machine). After that, 22.5 parts by weight of water was added and mixed.

Then, 60 parts by weight of Hachimi-jio-gan extract powder was added and the resultant was subjected to agitation granulation for 9 minutes. After the granulation, the resultant was dried and subjected to regularization.

Further, 0.16 part by weight of magnesium stearate was added to form granules for tableting, and circular tablets were produced from the granules using a tableting machine.

The tablets were subjected to the disintegration test described in Japan Pharmacopoeia. As a result, disintegration times for the six tablets were 27.8 minutes, 28.6 minutes, 28.7 minutes, 29.7 minutes, 30.3 minutes, and 30.9 minutes, respectively, with the average of the six tablets being 29.3 minutes.

Embodiment 8

A granulated product containing extract powder of Bofu-tsusho-san serving as the Chinese orthodox medicine was produced as follows.

10 parts by weight of calcium silicate (trade name: FLORITE-RE) and 17 parts by weight of crosslinked polyvinylpyrrolidone (trade name: Kollidon CL) were sifted and charged into Vertical Granulator (granulating machine). After that, 17.5 parts by weight of water was added thereto and mixed.

Then, 60 parts by weight of Bofu-tsusho-san extract powder was added and the resultant was subjected to agitation granulation for 8 minutes. After the granulation, the resultant was dried and subjected to regularization.

Further, 0.5 part by weight of magnesium stearate was added to form granules for tableting, and circular tablets were produced from the granules using a tableting machine.

The tablets were subjected to the disintegration test described in Japan Pharmacopoeia. As a result, disintegration times for the six tablets were 5.7 minutes, 6.2 minutes, 6.6 minutes, 6.7 minutes, 7.0 minutes, and 7.5 minutes, respectively, with the average of the six tablets being 6.6 minutes.

### COMPARATIVE EXAMPLE 2

In Comparative Example 2 for Embodiment 8, commercially available Bofu-tsusho-san extract tablets were subjected to the disintegration test described in Japan Pharmacopoeia. As a result, average disintegration times for the six tablets were as follows.

Product C: 35 minutes

Product D: 40 minutes

Product E: 38 minutes.

Embodiment 9

A granulated product containing extract powder of Boi-ogi-to serving as the Chinese orthodox medicine was produced as follows.

10 parts by weight of calcium silicate (trade name: FLORITE-RE) and 18 parts by weight of crosslinked polyvinylpyrrolidone (trade name: Kollidon CL) were sifted and charged into Vertical Granulator (granulating machine). After that, 20 parts by weight of water was added thereto and mixed.

Then, 60 parts by weight of Boi-ogi-to extract powder was added and the resultant was subjected to agitation granulation for 8 minutes. After the granulation, the resultant was dried and subjected to regularization.

Further, 0.5 part by weight of magnesium stearate was added to form granules for tableting, and circular tablets were produced from the granules using a tableting machine.

The tablets were subjected to the disintegration test described in Japan Pharmacopoeia. As a result, disintegration times for the six tablets were 5.7 minutes, 6.3 minutes, 7.0 minutes, 7.5 minutes, 7.7 minutes, and 7.9 minutes, respectively, with the average of the six tablets being 7.0 minutes.

### COMPARATIVE EXAMPLE 3

In Comparative Example 3 for Embodiment 9, commercially available Hachimi-jio-gan extract tablets were subjected to the disintegration test described in Japan Pharmacopoeia. As a result, average disintegration time for the six tablets was as follows.

Product-F: 32 minutes.

Embodiment 10

A granulated product containing extract powder of Sho-seiryu-to serving as the Chinese orthodox medicine was produced as follows.

10 parts by weight of calcium silicate (trade name: FLORITE-RE) and 21 parts by weight of crosslinked polyvinylpyrrolidone (trade name: Kollidon CL) were sifted and charged into Vertical Granulator (granulating machine). After that, 20 parts by weight of water was added thereto and mixed.

Then, 60 parts by weight of Sho-seiryu-to extract powder was added and the resultant was subjected to agitation granulation for 3 minutes. After the granulation, the resultant was dried and subjected to regularization.

Further, 1.4 parts by weight of magnesium stearate was added to form granules for tableting, and circular tablets were produced from the granules using a tableting machine.

The tablets were subjected to the disintegration test described in Japan Pharmacopoeia. As a result, disintegration times for the six tablets were 6.0 minutes, 6.4 minutes, 7.0 minutes, 7.1 minutes, 7.9 minutes, and 8.1 minutes, respectively, with the average of the six tablets being 6.8 minutes.

### COMPARATIVE EXAMPLE 4

In Comparative Example 4 for Embodiment 10, commercially available Sho-seiryu-to extract tablets were subjected to the disintegration test described in Japan Pharmacopoeia. As a result, average disintegration time for the six tablets was as follows.

Product G: 19 minutes.

Embodiment 11

A granulated product containing extract powder of Hochu-ekki-to serving as the Chinese orthodox medicine was produced as follows.

10 parts by weight of calcium silicate (trade name: FLORITE-RE) and 16 parts by weight of crosslinked polyvinylpyrrolidone (trade name: Kollidon CL) were sifted and charged into Vertical Granulator (granulating machine). After that, 22.5 parts by weight of water was added thereto and mixed.

Then, 60 parts by weight of Hochu-ekki-to extract powder was added and the resultant was subjected to agitation granulation for 12 minutes. After the granulation, the resultant was dried and subjected to regularization.

Further, 0.8 part by weight of magnesium stearate was added to form granules for tableting, and circular tablets were produced from the granules using a tableting machine.

The tablets were subjected to the disintegration test described in Japan Pharmacopoeia. As a result, disintegration times for the six tablets were 5.7 minutes, 7.1 minutes, 7.2 minutes, 7.5 minutes, 7.6 minutes, and 7.6 minutes, respectively, with the average of the six tablets being 7.3 minutes.

### COMPARATIVE EXAMPLE 5

In Comparative Example 5 for Embodiment 11, commercially available Hochu-ekki-to extract tablets were subjected to the disintegration test described in Japan Pharmacopoeia. As a result, average disintegration time for the six tablets was as follows.

Product H: 32.1 minutes.

Embodiment 12

A granulated product containing a crude drug extract was produced as follows.

10 parts by weight of calcium silicate (trade name: FLORITE-RE) and 16 parts by weight of crosslinked polyvinylpyrrolidone (trade name: Kollidon CL) were sifted and charged into Vertical Granulator (granulating machine). After that, 22.5 parts by weight of water was added thereto and mixed.

Then, 60 parts by weight of extract powders obtained from Paeoniae Radix, Asparagi Radix, Cinnamomi Cortex, Cnidii Rhizoma, Nupharis Rhizoma, Atractylodis Lanceae Rhizoma, Caryophylli Flos, Ginseng Radix, Poria, Glycyrrhizae Radix, Rhei Rhizoma, and Arecae Semen was added and the resultant was subjected to agitation granulation for 12 minutes. After the granulation, the resultant was dried and subjected to regularization.

Further, 0.8 part by weight of magnesium stearate was added to form granules for tableting, and circular tablets were produced from the granules using a tableting machine.

The tablets were subjected to the disintegration test described in Japan Pharmacopoeia. As a result, disintegration times for the six tablets were 6.5 minutes, 7.0 minutes, 8.0 minutes, 8.5 minutes, 9.0 minutes, and 9.5 minutes, respectively, with the average of the six tablets being 8.1 minutes.

Embodiment 13

A granulated product containing an extract powder derived from a natural substance was produced as follows.

10 parts by weight of calcium silicate (trade name: FLORITE-RE) and 16 parts by weight of crosslinked polyvinylpyrrolidone (trade name: Kollidon CL) were sifted and charged into Vertical Granulator (granulating machine). After that, 22.5 parts by weight of water was added thereto and mixed.

Then, 60 parts by weight of extract powders obtained from Silybum marianum and curcuma (Turmeric) was added and the resultant was subjected to agitation granulation for 12 minutes. After the granulation, the resultant was dried and subjected to regularization.

Further, 0.8 part by weight of magnesium stearate was added to form granules for tableting, and circular tablets were produced from the granules using a tableting machine.

The tablets were subjected to the disintegration test described in Japan Pharmacopoeia. As a result, disintegration times for the six tablets were 7.0 minutes, 8.0 minutes, 8.5 minutes, 9.0 minutes, 9.5 minutes, and 10.0 minutes, respectively, with the average of the six tablets being 8.7 minutes.

Embodiment 14

A granulated product containing an extract powder derived from a natural substance was produced as follows.

10 parts by weight of calcium silicate (trade name: FLORITE-RE) and 16 parts by weight of crosslinked polyvinylpyrrolidone (trade name: Kollidon CL) were sifted and charged into Vertical Granulator (granulating machine). After that, 22.5 parts by weight of water was added thereto and mixed.

Then, 60 parts by weight of an extract powder obtained from Saw palmet to serving as a natural substance was added and the resultant was subjected to agitation granulation for 12 minutes. After the granulation, the resultant was dried and subjected to regularization.

Further, 0.8 part by weight of magnesium stearate was added to form granules for tableting, and circular tablets were produced from the granules using a tableting machine.

The tablets were subjected to the disintegration test described in Japan Pharmacopoeia. As a result, disintegration times for the six tablets were 3.0 minutes, 4.0 minutes, 5.0 minutes, 5.5 minutes, and 6.0 minutes, respectively, with the average of the six tablets being 4.8 minutes.

### INDUSTRIAL APPLICABILITY

As described above, the present invention has the effects of: enabling practical granulation for producing the granulated product having a high content of the Chinese orthodox medicine extract, the crude drug extract, or the extract derived from a natural substance using the high speed agitation granulating machine; and also of enabling production of tablets having satisfactory disintegrating property having a high content of the Chinese orthodox medicine extract, the crude drug extract, or the extract derived from a natural substance from the granulated product.

1. A method of producing a granulated product, characterized by comprising: adding water to calcium silicate and a disintegrant; uniformly mixing and dispersing the water; and adding a Chinese orthodox medicine extract powder to the resultant with agitating and granulating.

2. A method of producing a granulated product, characterized by comprising: adding water to calcium silicate and a disintegrant; uniformly mixing and dispersing the water; and adding a crude drug extract powder to the resultant with agitating and granulating.

3. A method of producing a granulated product characterized by comprising: adding water to calcium silicate and a disintegrant; uniformly mixing and dispersing the water; and adding an extract powder derived from a natural substance to the resultant with agitating and granulating.

4. A method of producing a granulated product according to claim 1, 2 or 3, wherein the disintegrant to be used is crosslinked polyvinylpyrrolidone.

5. A method of producing a granulated product according to claim 1, 2 or 3, characterized in that 1 to 50 parts by weight of the disintegrant is added to 10 parts by weight of the calcium silicate.

6. A method of producing a granulated product according to claim 1, 2 or 3, characterized in that 7 to 30 parts by weight of the disintegrant is added to 10 parts by weight of the calcium silicate.

7. A method of producing a granulated product according to claim 1, 2 or 3, characterized in that 2 to 50 parts by weight of water is added to 10 parts by weight of the calcium silicate.

8. A method of producing a granulated product according to claim 1, 2 or 3, characterized in that 5 to 25 parts by weight of water is added to 10 parts by weight of the calcium silicate.

9. A method of producing a granulated product according to claim 1, 2 or 0.3, characterized in that an aqueous solution containing 30 wt % or less of ethanol is used in place of water.

10. A method of producing a granulated product according to claim 9, characterized in that 2 to 50 parts by weight of the aqueous solution containing ethanol is added to 10 parts by weight of the calcium silicate.

11. A method of producing a granulated product according to claim 10, characterized in that 5 to 25 parts by weight of the aqueous solution containing ethanol is preferably added to 10 parts by weight of the calcium silicate.

12. A method of producing a tablet, characterized in that in claim 1, 2 or 3, a weight composition of the extract in a tablet is 30 to 90 wt %.

13. A method of producing a granulated product comprising: adding water to a mixture of calcium silicate and a portion of a disintegrant; uniformly mixing and dispersing the water; adding a Chinese orthodox medicine extract powder to the resultant; agitating and granulating the resultant to obtain the granulated product; and adding a remainder of the disintegrant to the granulated product.

14. A method of producing a granulated product comprising: adding water to a mixture of calcium silicate and a portion of a disintegrant; uniformly mixing and dispersing the water; adding a crude drug extract powder; agitating and granulating the resultant to obtain the granulated product; and adding a remainder of the disintegrant to the granulated product.

15. A method of producing a granulated product comprising: adding water to a mixture of calcium silicate and a portion of a disintegrant; uniformly mixing and dispersing the water; adding an extract powder derived from a natural substance; agitating and granulating the resultant to obtain the granulated product; and adding a remainder of the disintegrant to the granulated product.

16. A method of producing a tablet, characterized in that in claim 13, 14 or 15, a weight composition of the extract in a tablet is 30 to 90 wt %.
